(19) United States
(12) Patent Application Publication
(10) Pub. No.: US 2023/0285073 A1
(43) Pub. Date: Sep. 14, 2023

(54) CATHETER

(71) Applicant: Japan Lifeline Co., Ltd., Tokyo (JP)

(72) Inventor: Kenji MORI, Tokyo (JP)

(21) Appl. No.: 18/179,849

(22) Filed: Mar. 7, 2023

(30) Foreign Application Priority Data
Mar. 11, 2022 (JP) 2022-037879

(51) Int. Cl. A61B 18/14

(52) U.S. Cl. CPC A61B 18/1492; A61B 2018/0022

(57) ABSTRACT

[Object]

Provided is a catheter that can enhance the smoothness of a lumen.

Solution

A balloon catheter includes: an outer shaft to be inserted into a body, the outer shaft including a main lumen that extends in an axial direction from a proximal end side toward a distal end side; and a second member exposed in a portion of an inner circumferential surface of the outer shaft to the main lumen and having frictional properties lower than those of a first member (an inner shaft) that forms the inner circumferential surface of the outer shaft. The balloon catheter further includes sub-shafts that include sub-lumens extending in the axial direction and that are formed of the second member, and a portion of outer circumferential surfaces of the sub-shafts is exposed in a portion of the inner circumferential surface of the outer shaft to the main lumen.

### TECHNICAL FIELD

The present disclosure relates to a catheter to be inserted into the body.

### BACKGROUND ART

A catheter is a medical tube to be inserted into the body for diagnosis or treatment. Of catheters, some catheter is provided with a balloon inserted into a tubular organ such as a blood vessel, a trachea, a digestive tract, a common bile duct, or a pancreatic duct inside the body, a connection portion thereof (inlet and outlet), a hole formed in the body for diagnosis or treatment (for example, a hole punctured from the stomach or duodenal bulb into the common bile duct) to expand or treat a target portion. Such a catheter is called a balloon catheter, and an example of the balloon catheter is disclosed in Patent Literature 1. An outer shaft of the balloon catheter of Patent Literature 1 includes a central lumen through which an inner shaft or the like is inserted, and a plurality of surrounding lumens that are disposed around the central lumen to be used in supply, discharge, or the like of fluid for inflating the balloon.

### CITATION LIST

### Patent Literature

Patent Literature 1: WO 2021/130877

### SUMMARY OF INVENTION

### Technical Problem

In the outer shaft of Patent Literature 1, the central lumen is formed of a tube of a polyetheretherketone resin (PEEK resin) excellent in mechanical properties, but its relatively high frictional properties are likely to cause difficulty inserting the inner shaft or the like into the central lumen.

The present disclosure has been made in view of such circumstances, and an object thereof is to provide a catheter that can enhance the smoothness of a lumen.

### Solution to Problem

In order to solve the problem described above, a catheter according to an aspect of the present invention includes: a first shaft configured to be inserted into a body, the first shaft including a first lumen that extends in an axial direction from a proximal end side toward a distal end side; and a second member forming, together with a first member that is a main constituent member of the first shaft, an inner circumferential surface defining the first lumen, the second member having frictional properties lower than frictional properties of the first member.

According to this aspect, the inner circumferential surface of the first shaft is formed of the low-frictional second member, and thus the smoothness of the first lumen can be enhanced.

### Advantageous Effects of Invention

According to a catheter of the present disclosure, the smoothness of a lumen can be enhanced.

### BRIEF DESCRIPTION OF DRAWINGS

FIG. 1 illustrates the entirety of a balloon catheter.

FIG. 2 is a perspective view illustrating a balloon when inflated.

FIG. 3 is a cross-sectional view of the balloon when inflated.

FIG. 4 is a partially enlarged view of a proximal end portion of the balloon.

FIG. 5 is a partially enlarged view of a distal end portion of the balloon.

FIG. 6 is a cross-sectional view of an outer shaft.

FIG. 7 is a view of a state where the balloon and the like are removed from the state of FIG. 4.

FIG. 8 schematically illustrates an example of a method of manufacturing an outer shaft.

FIG. 9 illustrates a modified example of a second member.

### DESCRIPTION OF EMBODIMENTS

Hereinafter, modes for carrying out the present invention (hereinafter also referred to as “embodiments”) will be described in detail with reference to the drawings. In the description and/or drawings, the same or equivalent constituent elements, members, and processing operations, and the like are denoted by the same reference numerals, and redundant descriptions are omitted. The scales and shapes of the illustrated parts are set for convenience to simplify the explanation and should not be construed in a limited manner unless otherwise specified. The embodiments are illustrative and do not limit the scope of the present invention in any way. Not all features or combinations of the features described in the embodiments are essential to the present invention.

Although the present disclosure can be applied to any type of catheter, a balloon catheter including a balloon with the surface on which a strip-shaped electrode to be applied with radio-frequency power (hereinafter, also abbreviated and referred to as radio frequency) is described as an example in the present embodiment. This balloon catheter is utilized in catheter ablation (hereinafter, also abbreviated and referred to as ablation), which is a method for the treatment of arrhythmia, or radio frequency ablation (RFA). The balloon inserted into an abnormal site (vessel itself or surrounding tissue around the focus or the like) in a vessel such as a blood vessel that is a cause of arrhythmia inflates with inflation fluid such as saline supplied thereinto and brings the electrode on the surface close to or in contact with the abnormal site. The abnormal site is cauterized by the radio frequency applied to the electrode in this state.

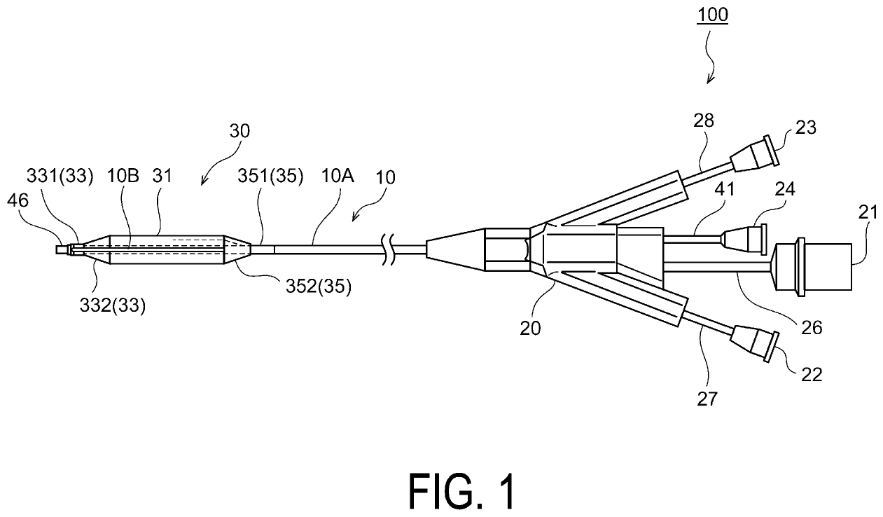

FIG. 1 illustrates the entirety of a balloon catheter 100 according to the present embodiment. The balloon catheter 100 includes an outer shaft 10 (hereinafter, also abbreviated and referred to as a shaft 10) as a first shaft to be inserted into the body, a handle portion 20 attached to the proximal end side of the outer shaft 10 or the outer side of the body (the right side in FIG. 1), and a balloon 30 attached to the distal end side of the outer shaft 10 or the inner side of the body (the left side in FIG. 1) to be inflatable with fluid supplied from the proximal end side of the outer shaft 10. The tubular outer shaft 10 having flexibility includes a tubular proximal end side shaft 10A extending from the handle portion 20 to a proximal end portion of the balloon 30, and a tubular distal end side shaft 10B communicating with the proximal end side shaft 10A and extending through the balloon 30 in an axial direction (a left-right direction in FIG. 1), and a distal end tip 46 attached to a distal end portion of the distal end side shaft 10B.

The balloon catheter 100 is utilized in ablation for cauterizing the focus. As described below, a strip-shaped electrode group is formed on the surface of the balloon 30 along the axial direction from the proximal end side toward the distal end side. The balloon 30 inserted into an abnormal site in a vessel such as a blood vessel inflates with inflation fluid such as saline supplied thereinto via the handle portion 20 and the outer shaft 10 and brings the electrode group on the surface close to or in contact with the abnormal site. The electrode group to which the radio frequency is applied in this state cauterizes the abnormal site.

An electrical connector 21, a fluid supply port 22, a fluid discharge port 23, and a device port 24 are disposed on the proximal end side of the handle portion 20. The electrical connector 21 is electrically connected to an electrode group on the surface of the balloon 30 by a conducting wire passing through an electrical cable 26, the handle portion 20, the shaft 10 (the proximal end side shaft 10A and/or the distal end side shaft 10B), and the balloon 30 from the proximal end side toward the distal end side. Therefore, the electrical connector 21 connected to a radio-frequency power supply (not illustrated) can apply radio frequency to the electrode group on the surface of the balloon 30. In addition, by connecting the electrical connector 21 to a control device or measurement device configured by a computer or the like, data such as a potential of a treatment site measured by the electrode group on the surface of the balloon 30 or a temperature of the treatment site measured by a temperature sensor (thermocouple) described below may be acquired.

The fluid supply port 22 supplies fluid for inflating the balloon 30, specifically, inflation fluid obtained by appropriately mixing a contrast agent into sterile distilled water or saline. The fluid supply port 22 is in communication from the proximal end side toward the distal end side with the interior of the balloon 30 via a flow path passing through a fluid supply tube 27, the handle portion 20, and the shaft 10 (sub-shafts 101 to 105 described below). The balloon 30 inflates when the fluid supply port 22 supplies the inflation fluid into the balloon 30. The fluid discharge port 23 discharges the inflation fluid from the interior of the balloon 30. The fluid discharge port 23 is in communication from the proximal end side toward the distal end side with the interior of the balloon 30 via a flow path passing through a fluid discharge tube 28, the handle portion 20, and the shaft 10 (sub-shafts 107 to 111 described below). The balloon 30 shrinks when the fluid discharge port 23 discharges the inflation fluid from the interior of balloon 30. Note that the inflation fluid in the balloon 30 may be distributed or circulated while the balloon 30 being inflated by simultaneously supplying the inflation fluid via the fluid supply port 22 into the balloon 30 and discharging the inflation fluid via the fluid discharge port 23 from the interior of the balloon 30.

A medical instrument provided at a distal end with a device such as a camera, forceps, or the like for diagnosis or treatment, or guide wire for guiding the balloon 30 to a treatment site is inserted into the device port 24. In the present embodiment, an example where the guide wire is inserted into the device port 24 is described. As described below, the proximal end side shaft 10A of the shaft 10 includes an outer shaft (hereinafter, may be denoted by reference numeral 10A for convenience) and an inner shaft 41, and the distal end side shaft 10B of the shaft 10 includes the inner shaft 41 (hereinafter, may be denoted by reference numeral 10B for convenience). The device port 24 is in communication with the inner shaft 41 extending in the axial direction between the proximal end and the distal end of the balloon catheter 100. The guide wire introduced from the device port 24 passes through the inner shaft 41 to extend out of the balloon catheter 100 from the distal end tip 46. Therefore, by inserting a distal end portion of the balloon catheter 100 from a proximal end portion of the guide wire inserted to a treatment site in advance, the balloon 30 can reach the treatment site while being guided by the guide wire. A substantially cylindrical space extending in the axial direction inside the tubular inner shaft 41 is referred to as a wire lumen or the like mainly when the guide wire is inserted, but hereinafter is collectively referred to as an inner lumen. Also, as described below, the inner shaft 41 is inserted into an outer lumen as a first lumen that is a substantially cylindrical space extending in the axial direction inside the outer shaft 10 (in particular, the outer shaft 10A).

The balloon 30 includes an intermediate portion 31 that can be inflated in a cylindrical shape by the inflation fluid supplied from the fluid supply port 22, and a distal end portion 33 attached at the distal end side of the intermediate portion 31 to the distal end side shaft 10B (inner shaft 41), and a proximal end portion 35 attached at the proximal end side of the intermediate portion 31 to the proximal end side shaft 10A (outer shaft 10). The intermediate portion 31 is a portion connecting, in the axial direction, the distal end portion 33 and the proximal end portion 35 that are attached to the shaft 10, and hereinafter is also referred to as a straight portion 31. The distal end portion 33 of the balloon 30 includes a distal end side neck portion 331 that is attached at the distal end side to the distal end side shaft 10B (inner shaft 41) or the distal end tip 46, and a distal end side tapered portion 332 (hereinafter, also referred to as a distal end side cone portion 332) that is formed in a tapered shape or a truncated cone shape from a distal end of the straight portion 31 toward the distal end side neck portion 331. The proximal end portion 35 of the balloon 30 includes a proximal end side neck portion 351 attached at the proximal end side to the outer circumference of the proximal end side shaft 10A (outer shaft 10), and a proximal end side tapered portion 352 (hereinafter, also referred to as a proximal end side cone portion 352) that is formed in a tapered shape or a truncated cone shape from a proximal end of the straight portion 31 toward the proximal end side neck portion 351.

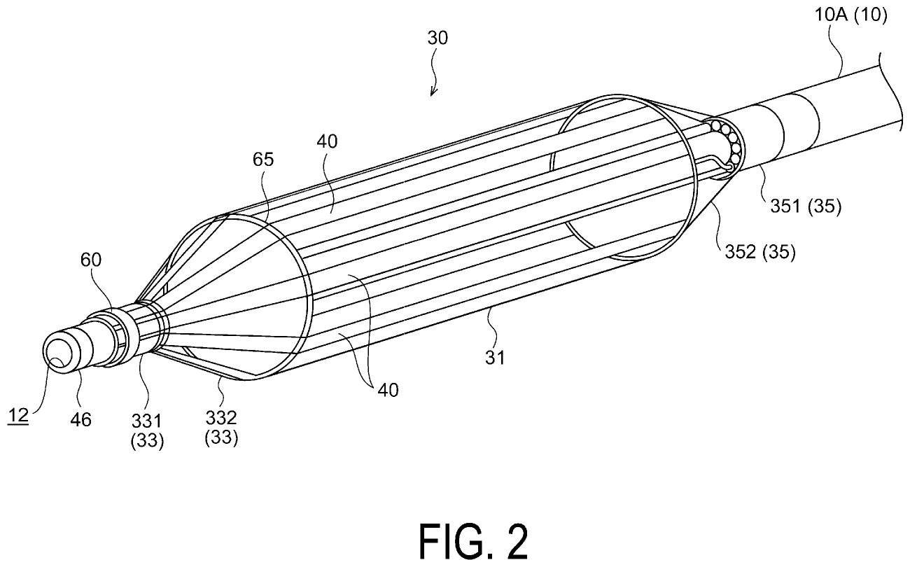

FIG. 2 is a perspective view illustrating the balloon 30 when inflated. A plurality of thin-film strip-shaped electrodes 40 are formed on the surface of the distal end portion 33 and the straight portion 31 of the balloon 30 along the axial direction from the distal end side toward the proximal end side. The strip-shaped electrodes 40 may be formed on the surface of the proximal end 35 of the balloon 30. The plurality of strip-shaped electrodes 40 are separated over the respective entire lengths in a circumferential direction of the balloon 30. Note that the plurality of strip-shaped electrodes 40 may be connected without gaps in the circumferential direction at at least the distal end side of the distal end side cone portion 332 and/or in the distal end side neck portion 331. In the illustrated example, the widths and intervals of the strip-shaped electrodes 40 in the circumferential direction are substantially constant, but may be different from each other.

To improve foldability of the balloon 30, the width of each of the strip-shaped electrodes 40 on the distal end portion 33 (and/or the proximal end portion 35) may be smaller than the width of each strip-shaped electrode 40 on the intermediate portion 31. For example, the width of the strip-shaped electrode 40 on the distal end side cone portion 332 may be monotonically decreased from the maximum width on the straight portion 31 to the minimum width on the distal end side neck portion 331.

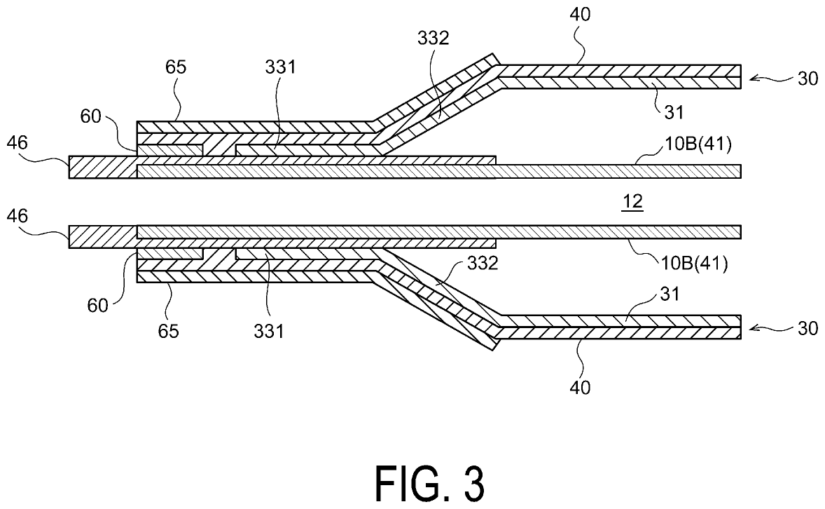

The configuration of the distal end portion 33 of the balloon 30 will be described also with reference to FIG. 3 schematically illustrating a cross-section taken along a plane including a central axis of the shaft 10 of FIG. 2. An inner lumen 12 (hereinafter, also referred to as a central lumen 12 or a main lumen 12) through which a device such as a guide wire can be inserted between the device port 24 and the distal end portion 33 is formed inside the distal end side shaft 10B (inner shaft 41) that extends through the interior of the balloon 30 in the axial direction. The outer diameter of the distal end side shaft 10B is, for example, 1.4 mm, and the inner diameter of the distal end side shaft 10B (the outer diameter of the central lumen 12) is, for example, 1.1 mm. The substantially cylindrical distal end tip 46 (a portion of the shaft 10) that covers and protects the distal end side shaft 10B, including the outer circumference thereof, is disposed at the distal end portion of the distal end side shaft 10B. The outer diameter of the distal end tip 46 is, for example, 2.0 mm, and the inner diameter of the distal end tip 46 is equal to that of the distal end side shaft 10B and is, for example, 1.1 mm. The distal end tip 46 is formed of a hard resin or the like. The device such as a guide wire that passes through the inner lumen 12 extends out of the balloon catheter 100 from the open ends of the distal end side shaft 10B and the distal end tip 46.

The distal end side neck portion 331 of the balloon 30 is attached to the proximal end side of the outer circumference of the distal end tip 46. Also, an annular ring electrode 60 as a circumferential electrode is disposed to the distal end side of the outer circumference of the distal end tip 46. The outer diameter of the ring electrode 60 is, for example, 2.22 mm, and the inner diameter of the ring electrode 60 is, for example, 2.08 mm. The strip-shaped electrode 40 such as silver (Ag) is formed on the outer circumferences of the ring electrode 60 and the balloon 30 by printing or the like to have a thickness of approximately 20 μm such that the gap between the distal end side neck portion 331 and the ring electrode 60 is filled. Note that the ring electrode 60 may be disposed on the outer periphery of the strip-shaped electrode 40 previously formed (FIG. 2 illustrates this example). The strip-shaped electrode 40 has a distal end substantially coinciding with a distal end of the ring electrode 60, and the distal end of the strip-shaped electrode 40 is located at a position retracted from a distal end of the distal end tip 46 toward the proximal end side. In other words, the distal end tip 46 further projects toward the distal end side than the strip-shaped electrode 40 and the ring electrode 60. Therefore, even when the distal end of the distal end tip 46 is brought into contact with an inner wall of a sheath (not illustrated) that guides the balloon 30 to a treatment site while storing the balloon 30 or with the body tissue, the strip-shaped electrode 40 and the ring electrode 60 can be prevented from being damaged.

An insulating coating 65 having a thickness between 10 μm and 20 μm is applied to the outer periphery of the strip-shaped electrode 40 from the distal end over the distal end side neck portion 331 and the distal end side cone portion 332. As just described, the strip-shaped electrode 40 is configured to be able to apply radio frequency to the treatment site at the outer circumference or the side surface of the straight portion 31 that has a stably inflated shape (cylindrical shape). Also, the ring electrode 60 connects the plurality of strip-shaped electrodes 40 in the circumferential direction at the distal end portion 33 of the balloon 30. Therefore, the plurality of strip-shaped electrodes 40 can apply radio frequency with the substantially same voltage and current to the treatment site. Consequently, even when the balloon 30 inserted into the body rotates in the circumferential direction, the radio frequency can be reliably applied to the treatment site. Note that, although not illustrated, the conducting wire passing from the electrical connector 21 through the electrical cable 26, the handle portion 20, the shaft 10 (sub-shaft 112 described below), and the balloon 30 is connected at the distal end portion of the balloon catheter 100 to the ring electrode 60 and/or the strip-shaped electrode 40. Therefore, the electrical connector 21 connected to the radio frequency power supply (not illustrated) can apply radio frequency to the strip-shaped electrode 40 on the surface of the balloon 30.

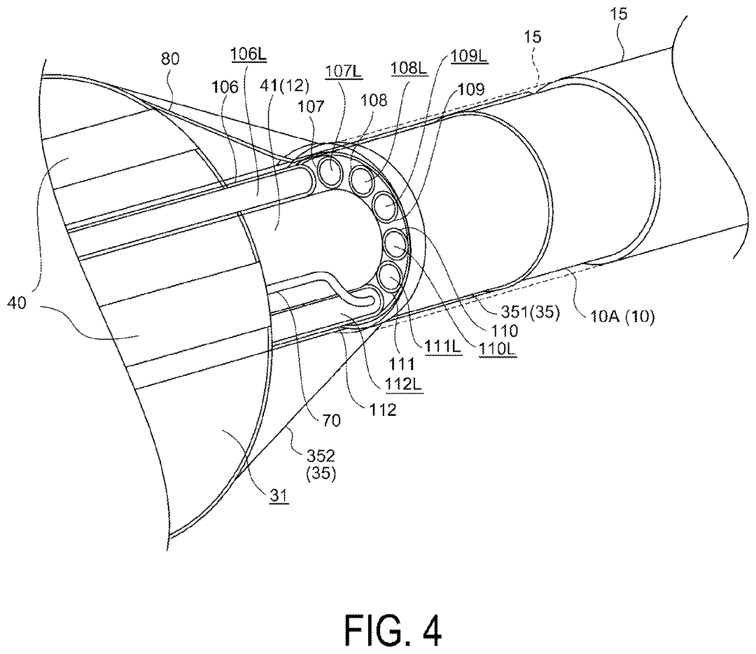

The configuration of the proximal end portion 35 of the balloon 30 will be described with reference to FIG. 4, which is a partially enlarged view of FIG. 2. In FIG. 4, the proximal end side neck portion 351 of the proximal end portion 35 of the balloon 30 is attached to the outer circumference of the proximal end side shaft 10A. Here, a portion of the outer circumference of the proximal end side shaft 10A to which the proximal end side neck portion 351 is attached is removed in advance by cutting or the like by the thickness (for example, 20 μm) of the balloon 30. Therefore, in a state where the proximal end side neck portion 351 is attached to the proximal end side shaft 10A, a step between an outer circumferential surface of the proximal end side neck portion 351 and an outer circumferential surface on the proximal end side of the proximal end side shaft 10A is minimized. The proximal end side neck portion 351 and the proximal end side shaft 10A adjacent to each other in the axial direction without a step as just described are covered by a tubular resin film 15 and thus are firmly fixed.

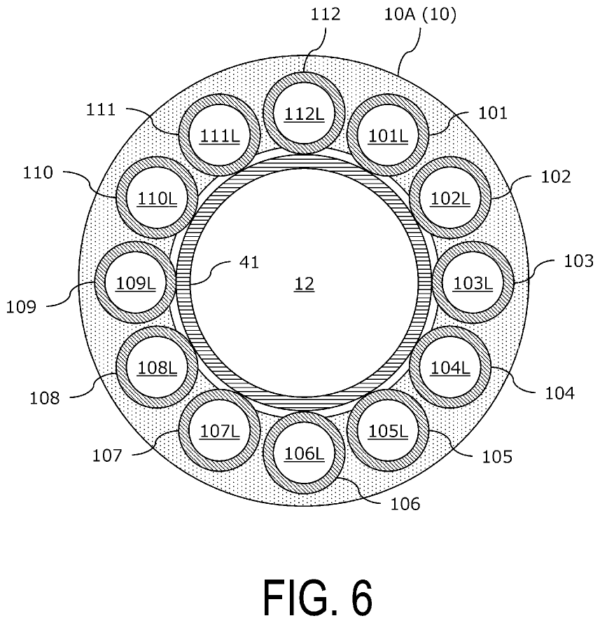

The outer shaft 10A as the first shaft includes the outer lumen as the first lumen through which the inner shaft 41 is inserted. In addition, as described above, the inner shaft 41 includes the inner lumen 12 through which a guide wire or the like is inserted. As illustrated in FIG. 6 described below, the outer lumen inside the outer shaft 10A and the inner lumen 12 inside the inner shaft 41 represent substantially the same space in a cross-section (strictly, the outer lumen is larger than the inner lumen 12 by the thickness of the inner shaft 41 and a below-described gap between an outer circumferential surface of the inner shaft 41 and an inner circumferential surface of the outer shaft 10A). Thus, the outer lumen is hereinafter denoted by the same numeral as the inner lumen 12 for convenience and is also referred to as the outer lumen 12. Also, the outer lumen 12 and the inner lumen 12 are collectively also referred to as the central lumen 12 or the main lumen 12.

In the example of FIG. 4, the inner lumen 12 or the main lumen 12 is formed, in the center of a cross-section of the proximal end side shaft 10A, which is orthogonal to the axial direction, as an internal space of the inner shaft 41 (FIG. 1) extending from the device port 24. In the cross-section of the proximal end side shaft 10A, which is orthogonal to the axial direction, a plurality of sub-lumens 101L to 112L (hereinafter, also referred to as surrounding lumens 101L to 112L) are disposed to surround the outer circumference of the main lumen 12 or the inner shaft 41. The plurality of sub-lumens 101L to 112L as second lumens are substantially cylindrical spaces formed by a plurality of tubular sub-shafts 101 to 112 (hereinafter, also referred to as surrounding shafts 101 to 112) as second shafts extending in the axial direction. Note that only the seven sub-lumens 106L to 112L and the seven sub-shafts 106 to 112 are illustrated in FIG. 4 (the five sub-lumens 101L to 105L and the five sub-shafts 101 to 105 are hidden and not visible).

Of the seven sub-lumens 106L to 112L illustrated in FIG. 4, the five sub-lumens 107L to 111L are opened into the balloon 30 at the proximal end portion 35 of the balloon 30, specifically, at a location near the boundary between the proximal end side neck portion 351 and the proximal end side tapered portion 352. Also, the proximal ends of the five sub-lumens 107L to 111L are connected to the fluid discharge port 23 (FIG. 1). Therefore, the inflation fluid inside the balloon 30 can be discharged from the fluid discharge port 23 through the five sub-lumens 107L to 111L. A conducting wire 70 having a proximal end connected to the electrical connector 21 is inserted into the sub-lumen 112L, and the conducting wire 70 extends to the ring electrode 60 and/or the strip-shaped electrode 40 illustrated in FIG. 3 or the like. Also, a thermocouple 80 having a proximal end connected to the electrical connector 21 (FIG. 1) is inserted into the sub-lumen 106L. The thermocouple 80 protrudes out of the sub-lumen 106L from a location on the proximal end side of a proximal end of the proximal end side neck portion 351 of the balloon 30 (the boundary with the proximal end side shaft 10A) while extending through the small-thickness balloon 30 (the proximal end side neck portion 351 and the proximal end side tapered portion 352) to a temperature measurement location in the intermediate portion 31. Note that the sub-lumens 106L, 112L, the thermocouple 80, and the conducting wire 70 are sealed or protected so as to prevent mixing or adhesion of the inflation fluid inside the balloon 30.

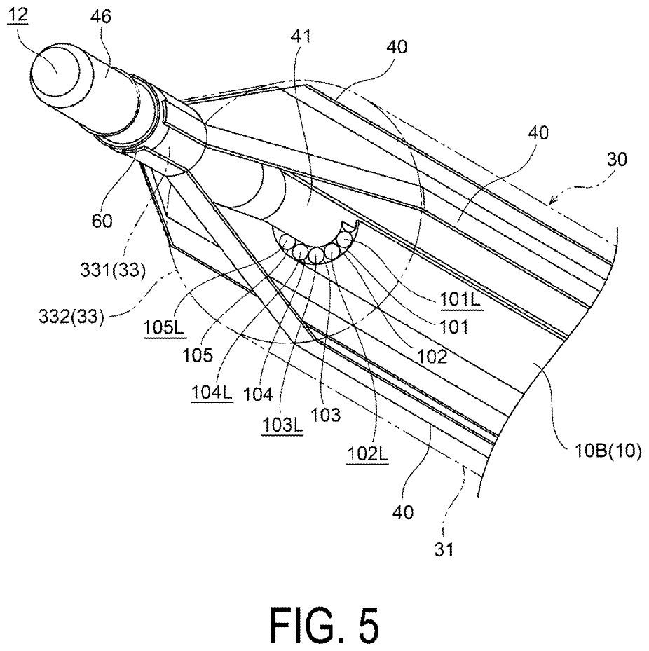

FIG. 5 is a partially enlarged view of the distal end portion 33 of the balloon 30. In FIG. 4 (the proximal end portion 35), the seven sub-lumens 106L to 112L of the twelve sub-lumens 101L to 112L are opened or terminated; therefore, only the remaining five sub-lumens 101L to 105L (and half of each of the sub-lumens 106L, 112L) extend in the distal end portion 33. Note that, as described below, the twelve sub-lumens 101L to 112L in total are located not overlapped with each other and are disposed at substantially equal intervals in a cross-section orthogonal to the axial direction.

The inner shaft 41 is inserted into the outer lumen 12 as the first lumen included in the distal end side shaft 10B. In the example of FIG. 5, the inner lumen 12 or the main lumen 12 is formed, in the center of a cross-section of the distal end side shaft 10B, which is orthogonal to the axial direction, as an internal space of the inner shaft 41 (FIG. 1) extending from the device port 24. In the cross-section of the distal end side shaft 10B, which is orthogonal to the axial direction, the plurality of sub-lumens 101L to 105L are disposed to surround the outer circumference of the main lumen 12 or the inner shaft 41 in a semi-circular shape. The plurality of sub-lumens 101L to 105L as the second lumens are substantially cylindrical spaces formed by the plurality of tubular sub-shafts 101 to 105 as the second shafts extending in the axial direction.

The five sub-lumens 101L to 105L are opened into the balloon 30 on the distal end side of the balloon 30, specifically, at a location near the boundary between the distal end side tapered portion 332 and the intermediate portion 31. Also, the proximal ends of the five sub-lumens 101L to 105L are connected to the fluid supply port 22 (FIG. 1). Therefore, the inflation fluid from the fluid supply port 22 can be supplied into the balloon 30 through the five sub-lumens 101L to 105L. The distal end side shaft 10B on the distal end side of the opening ends of the five sub-lumens 101L to 105L is formed only by the inner shaft 41. As described with respect to FIG. 3, the inner shaft 41 or the distal end side shaft 10B extends through the interior of the balloon 30 in the axial direction and thus forms, together with the distal end tip 46, the open end at the distal end of the balloon catheter 100. As just described, the balloon 30 attached to the distal end side of the shaft 10 as the first shaft can be inflated by the fluid supplied from the proximal end side of the sub-shafts 101 to 105 as the second shafts into the sub-lumens 101L to 105L as the second lumens.

Next, the relationship between the main lumen 12 in the center in the cross-section of the outer shaft 10, which is orthogonal to the axial direction and the twelve sub-lumens 101L to 112L around the main lumen 12 will be described with reference to FIG. 6. FIG. 6 illustrates a cross-section of the proximal end side shaft 10A of the outer shaft 10 in which any of the sub-lumens 101L to 112L is not terminated, for example, a cross-section of the proximal end side shaft 10A on the proximal end side of the proximal end of the proximal end side neck portion 351 of the balloon 30 (the boundary with the proximal end side shaft 10A) in FIG. 4. Note that the tubular resin film 15 covering the outer circumference of the proximal end side shaft 10A is not illustrated.

The twelve sub-lumens 101L to 112L are disposed to surround the outer circumference of the central main lumen 12 in a circle. Each of the sub-lumens 101L to 112L is a substantially cylindrical space inside each of the tubular sub-shafts 101 to 112 extending in the axial direction (direction orthogonal to a plane of paper in FIG. 6). In the illustrated example, the diameters, cross-sectional areas, and shapes of all of the sub-lumens 101L to 112L are substantially equal but may be different from each other. Additionally, the diameter and cross-sectional area of each sub-lumen 101L to 112L are smaller than those of the main lumen 12, but the diameter and cross-sectional area of at least one of the sub-lumens 101L to 112L may be larger than those of the main lumen 12. The twelve sub-lumens 101L to 112L and the twelve sub-shafts 101 to 112 are disposed at substantially equal intervals along a circumferential direction about the central axis of the main lumen 12 or the outer shaft 10. In other words, the intervals in the circumferential direction between the twelve sub-lumens 101L to 112L and the twelve sub-shafts 101 to 112 in the cross section of FIG. 6 are substantially constant. Note that when the plurality of intervals are substantially constant, the difference between any two intervals is less than 10% of an average value of all of the intervals. Here, the sub-shafts 101 to 112 respectively forming the sub-lumens 101L to 112L are not circumscribed with each other in the cross section of FIG. 6. As described below, a first member entered inside through gaps between the sub-shafts 101 to 112 forms a portion of the inner circumferential surface of the outer shaft 10, thus the main lumen 12 as illustrated is formed.

Each of the sub-shafts 101 to 112 as the second shafts is formed of a second member having lower frictional properties than those of the first member forming the inner circumferential surface of the outer shaft 10 as the first shaft. The second member forms an inner circumferential surface that defines the outer lumen 12 or the main lumen 12 as the first lumen together with the first member, which is a main constituent member of the outer shaft 10 as the first shaft. Here, the main constituent member is a member that forms a majority of the volume of the outer shaft 10. The first member (main constituent member of the outer shaft 10) is formed of, for example, nylon or urethane, and the second member (each sub-shaft 101 to 112) is formed of, for example, a fluororesin such as polytetrafluoroethylene (PTFE) or perfluoroalkoxy alkane (PFA). The coefficient of dynamic friction of the second member is preferably between 0.02 and 0.50 and is more preferably between 0.03 and 0.09.

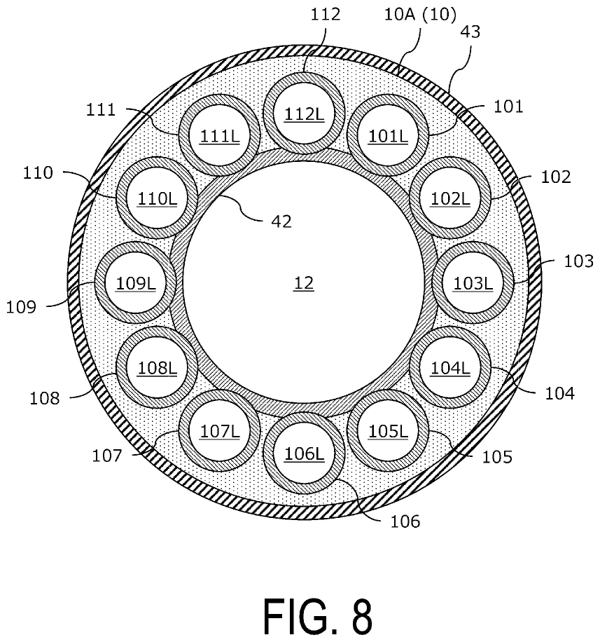

A portion of the outer circumferential surface of each sub-shaft 101 to 112 is exposed in a portion of the inner circumferential surface of the outer shaft 10 to the outer lumen 12 or the main lumen 12. In particular, in the illustrated example, the second member such as a fluororesin forming the outer circumferential surface of each sub-shaft 101 to 112 projects into the main lumen 12 from the first member forming the inner circumferential surface of the outer shaft 10. With these configurations, the inner shaft 41 is smoothly inserted into the outer lumen 12 or the main lumen 12 while being in contact with the low-frictional second member (each sub-shaft 101 to 112). Additionally, the first member having frictional properties higher than those of the second member is disposed between a plurality of the low-frictional second members (the sub-shafts 101 to 112) disposed along the circumferential direction of the inner circumferential surface of the outer shaft 10, and thus the smoothness of the outer lumen 12 or the main lumen 12 with respect to the inner shaft 41 can be prevented from being excessively high. Here, the proportion of the low-frictional second member (each of the sub-shafts 101 to 112) in the outer circumference of the main lumen 12 is optional but is preferably 30% or more in order to enhance the smoothness. In this case, the proportion of the high-frictional first member (main constituent member of the outer shaft 10) in the outer circumference of the main lumen 12 is less than 70%. Additionally, in order to enhance the smoothness of the main lumen 12, the proportion of the second member is preferably as high as possible, but in practice, an upper limit thereof is approximately 50% in light of the stability, efficiency, or the like in manufacturing described below with respect to FIG. 8.

In a known catheter such as that described in Patent Literature 1, a plurality of sub-shafts forming a plurality of sub-lumens are disposed around a main shaft forming a main lumen; however, in FIG. 6, it is unnecessary to provide an independent main shaft for forming the outer lumen 12 or the main lumen 12. Therefore, the outer lumen 12 or the main lumen 12 can be made larger than the known catheter. Alternatively, the plurality of sub-shafts can be brought closer to the outer lumen 12 or the main lumen 12 having the same or similar size as a known outer lumen or a known main lumen, and thus the diameter of the outer shaft 10 can be reduced compared to that of a known outer shaft.

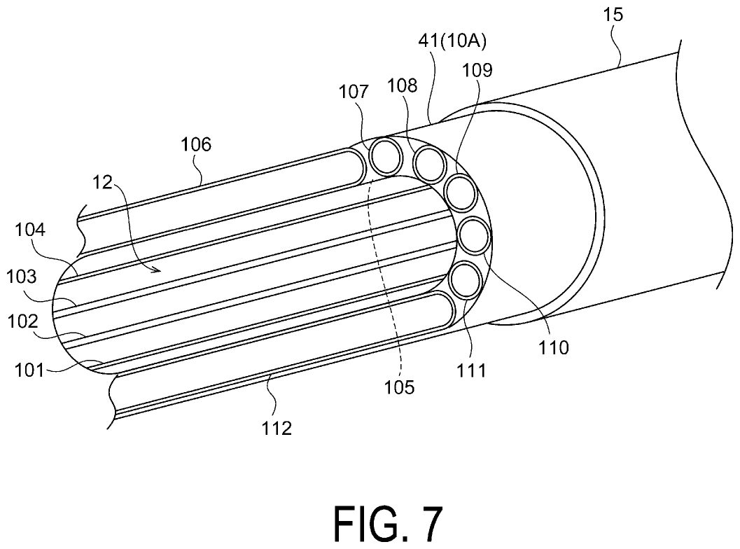

As schematically illustrated in FIG. 7 in which the balloon 30, the inner shaft 41, the conducting wire 70, the thermocouple 80, and the like are removed from FIG. 4, the low-frictional second members (the seven sub-shafts 101 to 106, 112 in FIG. 7) exposed into the outer lumen 12 or the main lumen 12 extend in the axial direction. Therefore, insertion of the inner shaft 41 into the outer lumen 12 or the main lumen 12 along the axial direction can be smoothly guided.

FIG. 8 schematically illustrates an example of a method of manufacturing the configuration of FIG. 6. A tubular or columnar first lumen forming member 42 has an outer circumferential surface that corresponds to the outer circumferential surface of the outer lumen 12 or the inner circumferential surface of the outer shaft 10. The twelve sub-shafts 101 to 112 are disposed at substantially equal intervals on the outer circumference of the first lumen forming member 42. Here, recessed portions into which the side surfaces of the respective sub-shafts 101 to 112 can be fitted may be disposed on the outer circumference of the first lumen forming member 42 such that the sub-shafts 101 to 112 can be appropriately disposed. Note that the material of the first lumen forming member 42 is optional, but is, for example, a fluororesin.

The first lumen forming member 42 and the twelve sub-shafts 101 to 112 are externally covered by a heat shrinkable tube 43. In this state, particles of a thermoplastic resin such as nylon or urethane, which is a main constituent member of the outer shaft 10 are fed into between the inner circumference of the heat shrinkable tube 43 and the outer circumference of the first lumen forming member 42 and the twelve sub-shafts 101 to 112 and are heated. The heated and melted thermoplastic resin enters inward through the gaps between the sub-shafts 101 to 112 and forms the inner circumferential surface of the outer shaft 10 along with the outer circumferential surface of each of the sub-shafts 101 to 112. Also, the heat shrinkable tube 43 heated to shrink forms the outer circumferential surface of the outer shaft 10. The configuration of FIG. 6 is achieved by removing the first lumen forming member 42 and the heat shrinkable tube 43 after cooling.

The present disclosure has been described above based on the embodiments. It should be understood by those skilled in the art that the embodiments are examples, that various modifications are possible in the combination of components and processing operations of the embodiments, and that such modifications are also within the scope of the present disclosure.

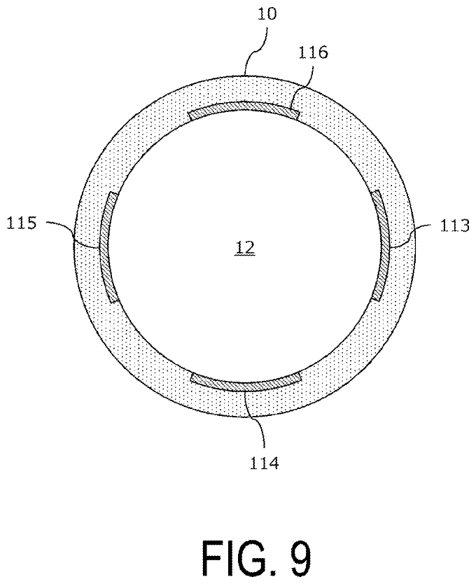

In the embodiment described above, the low-frictional second member exposed in a portion of the inner circumferential surface of the first shaft (the outer shaft 10) to the first lumen (the outer lumen 12 or the main lumen 12) is formed of the outer circumferential surfaces of the sub-shafts 101 to 112 as the second shafts, but the configuration of the low-frictional second member is not limited thereto. For example, as schematically illustrated in FIG. 9, one or a plurality of low-frictional second members 113 to 116 may be disposed along the circumferential direction on the inner circumferential surface of the high-frictional outer shaft 10. In this example, the second members 113 to 116 form an inner circumferential surface that defines the outer lumen 12 along with the first member that is a main constituent member of the outer shaft 10. Note that the inner shaft 41 in FIG. 6 is not illustrated in FIG. 9. Additionally, the second members 113 to 116 may extend in the axial direction from the proximal end toward the distal end of the shaft 10. Alternatively, the second members 113 to 116 may be interspersed in the axial direction instead of extending in the axial direction, or may be formed in a spiral manner about the axial direction.

### REFERENCE SIGNS LIST

10 Outer shaft
10A Proximal end side shaft
10B Distal end side shaft
12 Main lumen
24 Device port
30 Balloon
41 Inner shaft
100 Balloon catheter
101 to 112 Sub-shaft
101L to 112L Sub-lumen
113 to 116 Second member

1. A catheter, comprising:
a first shaft configured to be inserted into a body, the first shaft including a first lumen that extends in an axial direction from a proximal end side toward a distal end side; and
a second member forming, together with a first member that is a main constituent member of the first shaft, an inner circumferential surface defining the first lumen, the second member having frictional properties lower than frictional properties of the first member.

2. The catheter according to claim 1, wherein the second member extends in the axial direction.

3. The catheter according to claim 2, wherein the second member extends in the axial direction from a proximal end toward a distal end of the first shaft.

4. The catheter according to claim 1, wherein a plurality of the second members are disposed along a circumferential direction of the inner circumferential surface.

5. The catheter according to claim 4, wherein intervals in the circumferential direction between the plurality of second members are substantially constant.

6. The catheter according to claim 1, wherein the second member projects into the first lumen from the first member.

7. The catheter according to claim 1, wherein the second member includes a fluororesin.

8. The catheter according to claim 1, further comprising a second shaft including a second lumen extending in the axial direction, the second shaft including the second member,
wherein a portion of an outer circumferential surface of the second shaft forms, together with the first member, the inner circumferential surface defining the first lumen.

9. The catheter according to claim 8, further comprising a balloon attached to a distal end side of the first shaft, the balloon configured to be inflatable by a fluid supplied from a proximal end side of the second shaft into the second lumen.
